(19) United States
(12) Patent Application Publication
(10) Pub. No.: US 2008/0290050 A1
(43) Pub. Date: Nov. 27, 2008

(54) Device, Kit to Filter Bone Marrow Aspirate

(75) Inventor: Geraldo Marins dos Reis, JR., Campinas (BR)

Correspondence Address: SUGHRUE MION, PLLC, 2100 PENNSYLVANIA AVENUE, N.W., SUITE 800, WASHINGTON, DC, 20037, US

(73) Assignee: GM DOS REIS JR INDUSTRIA E COMERCIO DE EQUIPAMENTOS MEDICOS, Campinas (BR)

(21) Appl. No.: 12/093,239

(22) PCT Filed: Nov. 11, 2005
(86) PCT No.: PCT/BR05/00233

§ 371 Date: Jul. 22, 2008

(51) Int. Cl. B01D 24/00; C12N 5/08; A61F 2/28

(52) U.S. Cl. 210/800; 210/348; 210/454; 210/436

(57) ABSTRACT

The present invention refers to a filtering device for bone marrow aspirate, a kit comprising said filtering device, a method to obtain a biocompatible material enriched with progenitor cells, the material per se obtained from said method and to the use of said material for bone treatment, both in humans and animals.

### FIELD OF THE INVENTION

The present invention refers to a filtering device for bone marrow aspirate, a kit comprising said device, a method to obtain biocompatible material enriched with progenitor cells and an enriched filtering element resulting of said method.

### BACKGROUND OF THE INVENTION

Methods to obtain biocompatible material enriched with progenitor cells for posterior grafting into human and animal patients have been object of a constant research.

Due to the growing demand for these materials, various devices and methods have been proposed. However, said methods and/or devices do not present satisfactory results, i.e., they are subject to risks of contamination and loss of the material, they use methods that could damage its integrity and are not fully efficient.

Document WO 02/40963 A2, published on May 23, 2003, discloses a β-tricalcium phosphate composite, a method for its preparation and a kit. The method basically consists in retaining the biocompatible material within the porous matrix, allowing its coagulation and grafting it into the patient. The porous matrix is accommodated within syringes, which aspirate the bone marrow material and retain the matter of interest, which is then applied to the patient by means of the syringe itself. The kit comprises a syringe to inject and/or to take off the material and the porous matrix.

U.S. Pat. No. 6,723,131 patent discloses a bone marrow graft containing a porous biocompatible implantable matrix and a clot material, wherein the bone marrow graft is enriched with a cell population. This document also discloses a kit to prepare said graft, wherein the kit comprises a recipient containing matrix and a syringe at each end, so that the material is introduced by an end and, at the other one, the movement of the syringe piston guarantees the flow of the material along the recipient containing the matrix, thus resulting in a matrix imbibed in the material which will be later grafted into the patient. It is important to point out that the matrix of said document may have various forms, including, chips, chunks or fragments.

Patents U.S. Pat. No. 5,824,084 and U.S. Pat. No. 6,049,026 disclose a method to prepare bone grafts and a device to prepare an implantable graft. It is also described a population enriched with progenitor cells from the connective tissue, the resulting graft, as well as a kit to obtain said graft. The kit comprises a reservoir to retain the aspirated bone marrow, wherein said reservoir has two ends with openings, a flow regulator and a porous biocompatible matrix which may be grafted to retain and pass marrow material by dipping said matrix.

A few products employing the technology as disclosed by these previous documents are already commercialized. Said products comprise various processes to supply pressure to the system so to guarantee the flow of marrow material along the biocompatible material. However, said systems, as well as the documents in the state of the art have a limited efficiency, since not all bone marrow materials effectively pass through the biocompatible material and also the used pressure damages progenitor cells, which are the final interest of this procedure. Progenitor cells may be even more damaged in systems including recirculation of the marrow material.

The development as reached by the inventors of the present invention has allowed obtaining a device that does not present the problems known by the state of the art.

Further objects, features, aspects and advantages of the present invention will appear more clearly by reading the description and the description of figures below.

### DESCRIPTION OF FIGURES

FIG. 1 shows a front cross-section view of the device (6) of the present invention as disassembled.

FIG. 2 shows a front cross-section view of the device (6) of the present invention as assembled.

FIG. 3 shows a cut front view and an upper view of the tub (10) of the device (6) of the present invention.

FIG. 4 shows a front cross-section view of variations of the device (6) of the present invention.

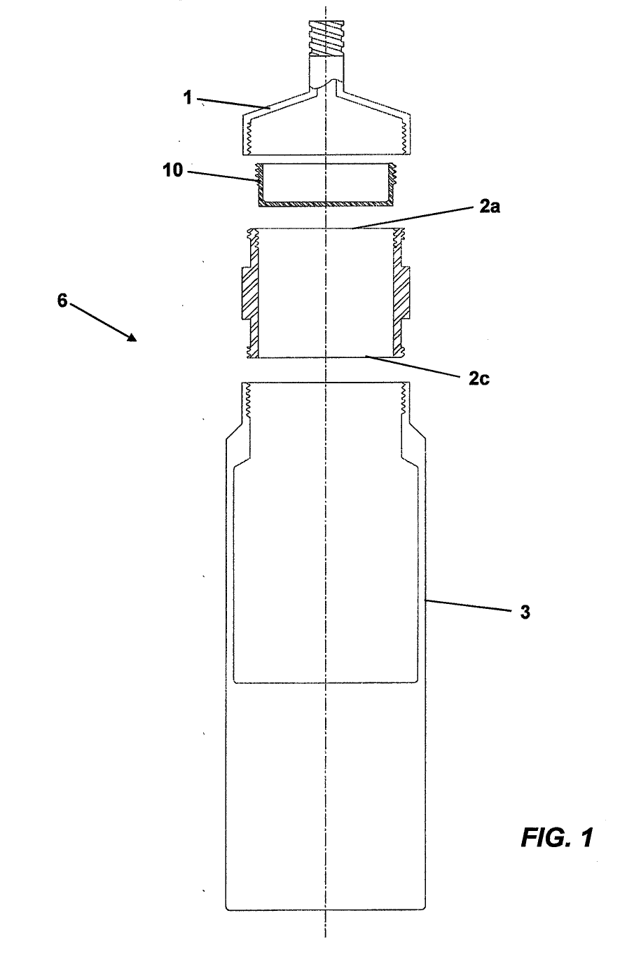

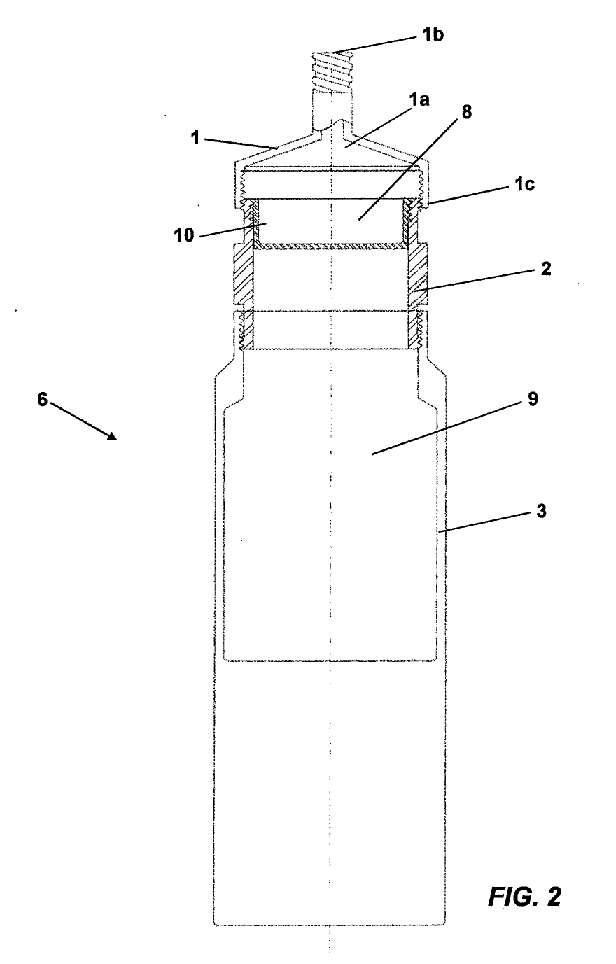

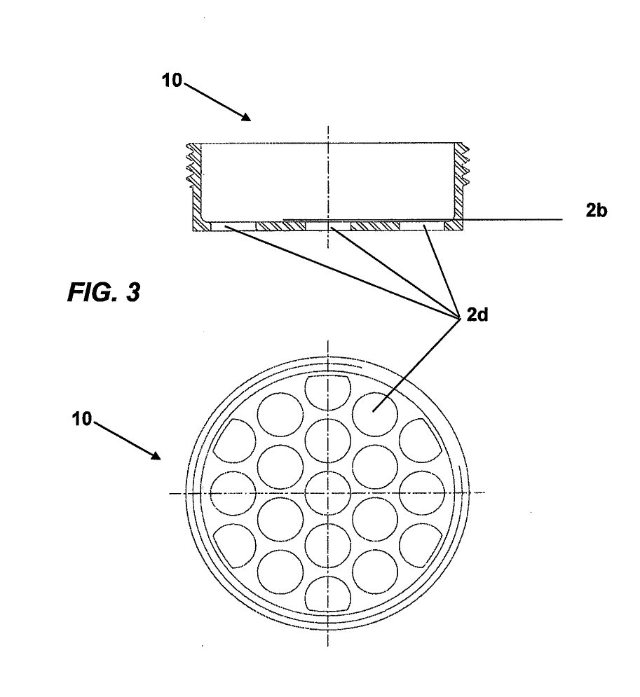

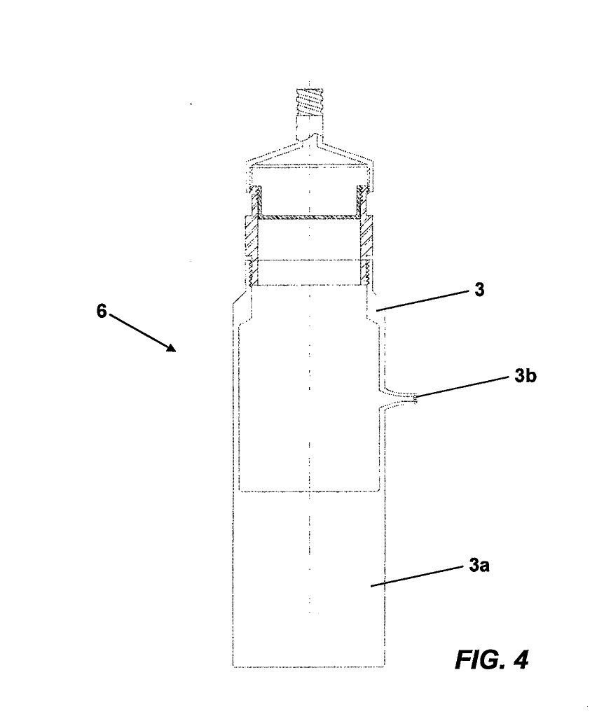

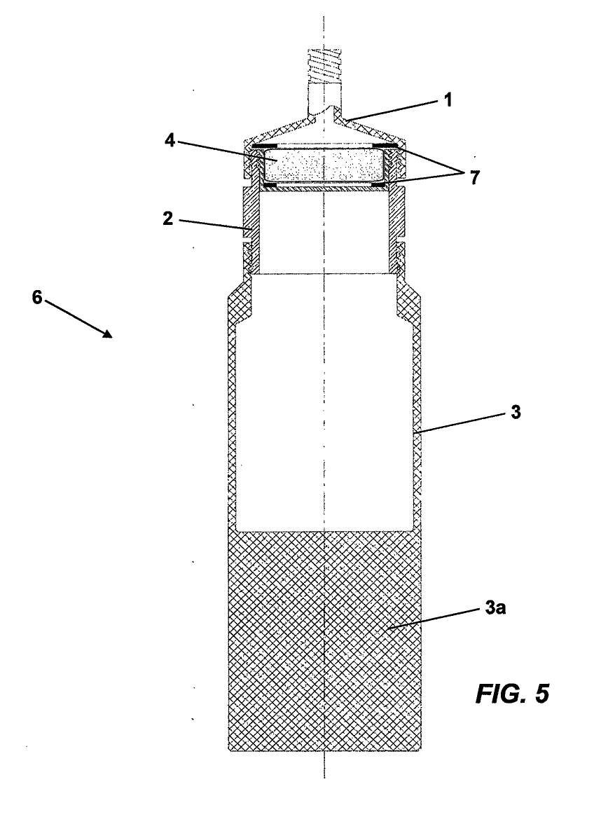

A FIG. 5 shows a front cross-section view of variations of a particular embodiment of the device (6) of the present invention.

### DESCRIPTION OF THE INVENTION

The technology used in the present invention is based on filtering a bone marrow aspirate, wherein stem cells, said progenitor cells from the bone marrow are retained within the filtering element (4) comprised in the device (6) of the present invention.

Said stem cells, when retained within the filtering element (4), are applied to a human or animal patient to promote bone growing at the place where it was inserted.

The device of the invention is very advantageous, since it provides full sealing to the filtering element thus avoiding contamination and loss of the material to be filtered and resulting in the full filtering through the biomaterial element. Furthermore, it allows filtering by means of a process that does not damage the integrity of the filtered material and the cells to be obtained.

Additionally, the device of the present invention is fully safe, not bringing in risks to the manipulator.

A first object of the present invention therefore refers to a filtering device for bone marrow aspirate (6) comprising a cover (1), a filtering component (2) and a recipient (3), wherein said filtering component (2) accommodates a filtering element (4) and at least one sealing element (7).

The cover (1), the filtering component (2) and the recipient (3) are connected by any means that guarantee the device integrity (6), such as threads, pressure fits or grooves and prominences. According to a preferential embodiment of the present invention, said elements are joined by means of threads.

Said cover (1) has an upper end (1a) provided with a beak or orifice (1b) and a lower end (1c) which remains in contact with the filtering component (2). The dimensions of the beak or orifice (1b) may vary according to the instrument used to aspirate and/or introduce marrow aspirate into the device (6) of the present invention.

The filtering component (2) comprises an upper cavity (2a) and a lower cavity (2c), which are formed by a tub (10), which base constitutes a wall (2b) provided with at least one opening (2d). Said upper cavity (2a) accommodates the filtering element (4), which is fixed by at least one sealing element (7). According to an alternative embodiment, the filtering component (2) is formed by one single piece presenting a division wall such as the wall (2b).

Dimension(s) of the upper (2a) and/or lower (2c) cavity(ies) may vary, notably the upper cavity (2a) may vary according to the volume of aspirated material to be filtered. The wall (2b) accommodates the filtering element (4) and the sealing element(s) (7) and should allow the passing of the material flow after filtering by means of at least one opening (2d).

Said wall (2b), being or not the base of the tub (10), may comprise at least one to a range of openings (2d), presenting various configurations such as a circle, rectangle, triangle, their combinations and others.

The sealing element (7) of the present invention is advantageous, since it safely fixes the filtering element (4), guaranteeing that all the aspirated bone marrow is effectively filtered, i.e., passes through the filtering element (4). Furthermore, the filtering mechanism, in opposition to the imbibing one previously employed, guarantees that most of the area of the biocompatible material is used by the cells of interest, more specifically progenitor cells. Said mechanism also guarantees that the cells of interest reach the most of the area of the biocompatible material used, including the central area, difficult to be reached by other similar methods. Said device avoids loss of bone marrow material and results in a more efficient process to obtain the homogeneously enriched filtering element.

Furthermore, its result is usually reached by only one passage of the bone marrow material by the filtering element (4), by using reduced pressure, equivalent e.g. to the required pressure to operate the syringe, thus resulting in a faster and more efficient process.

According to the present invention, said sealing element (7) may be located over or under the filtering element (4). Alternatively, said sealing element may involve or circum the filtering element (4).

According to a particular embodiment of the present invention, the filtering element (4) is fixed by two sealing elements (7), wherein the first element is assembled at the upper portion and the second element is assembled at the lower portion of the filtering element (4).

According to another embodiment of the present invention, the location of the sealing element may comprise a combination of the different arrangements above. As an example, said filtering element (4) may be assembled between two sealing elements (7) and yet be involved or circum by a third sealing element (7).

Said sealing element (7) is made of a biocompatible material, particularly of an elastomeric material, particularly silicone. Furthermore, the shape of said element (7) may vary according to the configuration of the filtering component (2) and the filtering element (4), since said element may comprise various shapes and/or dimensions according to the desired application.

Therefore, the present invention allows using various filtering elements (4), both regarding their composition as well as their shape and dimensions.

The filtering element (4) of the present invention should allow the bone marrow aspirated from bone marrow to be filtered and the cells of interest to be retained. Said filtering element is preferably composed by a biocompatible graft material, more particularly essentially constituted by β-tricalcium phosphate and optionally comprising from 1 to 90% of hydroxyapatite in relation to the total composition of said filtering element (4).

In a particular embodiment of the invention, the pore sizes of said filtering element (4) are comprised between 1 μm and 1000 μm.

The filtering element (4) of the present invention may present any form, such as powder, chips, granules, paste, tablets and block or a mixture thereof. The blocks and tablets useful according to the present invention may present a variety of geometric shapes as for example, cylindrical, disks, parallelepipeds, cubicles, prismatic, oval, and spherical. Particularly, the tablets have the shape of a disk. Furthermore, said filtering material (4) may have any dimensions, depending on the used device (6). In a preferred embodiment of the present invention, said filtering element has 2.8 cm diameter and 1 cm thickness.

Optionally, said filtering element (4) may be any matrix as known by a skilled in art, such as the ones designated as Vitoss®, Healos®, or any other appropriate matrix for that purpose.

According to the present invention, the recipient (3) may be cylindrical, rectangular, square, oval, spherical, pyramidal or others. According to a specific embodiment of the invention, the recipient (3) is cylindrical.

According to a particular embodiment of the present invention, the recipient (3) may have an anatomic shape or even a strap or handle, to help in its handling.

Once mounted, the device (6) has an upper chamber (8) formed by the cavity of the cap (1) and the tub cavity (10) located within the filtering element (2) and a lower chamber (9) corresponding to the cavity of the recipient (3) itself.

According to an even more particular embodiment of the present invention, the volume ratio between chambers (8):(9) is about 0.5:15, particularly of 1:13.6.

According to a variation of the present invention, the recipient (3) comprises an opening (3b), useful mainly when the device is handled by a non specialized professional, avoiding excessive pressure that could damage the cells, or the introduction of an instrument for getting the already filtered aspirate for eventual reprocess. Furthermore, in a preferred embodiment the device presents two openings (3b), each one for a specific purpose, i.e., air outlet or needle inlet. Said openings may be present at any face, level or angle of the recipient.

According to another variation of the present invention, said recipient (3) comprises a holding base (3a). Said holding base offers more safety when handling said device.

According to the present invention, the holding base (3a) may be cylindrical, rectangular, square, pyramidal, among others, not necessarily having the same shape of the recipient (3). According to a particular embodiment of the present invention, the base (3a) is cylindrical.

According to an even more particular embodiment of the present invention, the height ratio between the recipient (3) and the base (3a) is about 1:0.2 a 1:0.8, particularly of about 1:0.43.

According to the present invention, the device (6) may be disposable or reusable, and a person skilled in the art will know how to define the most appropriate material and, where required, the form of its sterilization. Preferably, the device is made of biocompatible material, particularly of polycarbonate (for example of Zelux).

A second object of the present invention refers to a kit to obtain a filtering element enriched with cells from the bone marrow comprising at least the following components:
i) a filtering device for bone marrow aspirate (6) as described by the present invention; and
ii) a filtering element (4).

The filtering device for bone marrow aspirate (6) as contained in the kit of the present invention is as previously described.

The kit of the present invention also comprises a filtering element (4).

According to another embodiment of the present invention, the kit additionally comprises (iii) one or more bone marrow aspiration devices. A suitable device for bone marrow aspiration is a cannula and trocar.

According to another embodiment of the present invention, the kit additionally comprises (iv) at least a syringe.

According to a preferred embodiment, the device comprises both (iii) and (iv) elements.

According to another embodiment of the present invention, the kit additionally comprises (v) a device for modeling the filtering element (4), according to the application desired, such as a template.

According to another embodiment of the present invention, the kit additionally comprises (vi) a device for fractioning of the filtering element (4), such as a scalpel.

According to another embodiment of the present invention, the kit additionally comprises (vii) an auxiliary device for placing and/or withdrawal of the filtering element (4), such as a clamp or forceps.

According to a preferred embodiment of the present invention, the device comprises the elements (v), (vi) and (vii) as described above.

Another object of the present invention is a method to obtain biocompatible graftable material enriched with progenitor cells from the bone marrow aspirate by applying the device (6). Said method comprises the following steps:
a) obtaining bone marrow aspirate;
b) filtering the bone marrow aspirate by introducing it within the filtering device (6), containing the filtering element (4), which is constituted by a biocompatible graft material; and
c) withdrawing said filtering element (4).

The obtainment of the aspirate from item (a) and the introduction thereof within the filtering device of item (b) are made by the use of the elements (iii) and (iv) described above.

According to the present invention, the steps (a) and (c) are carried out at least one time. According to a particular embodiment of the invention, the bone marrow material is processed more times, i.e., step (b) may be reprocessed at least once, by reintroducing it within the device of the already filtered aspirate, which is inside of the recipient (3).

The present invention also refers to the enriched biocompatible material obtained according to the method of the present invention.

Another object of the present invention is the use of said enriched biocompatible material of the present invention for bone treatment, both in humans and animals.

As well known by a person skilled in the art, numerous modifications and variations of the invention are possible in the light of the above teachings, not diverging from its scope of protection, as defined by the attached claims.

1-48. (canceled)

49. A filtering device for bone marrow aspirate, comprising a cover, a filtering component and a recipient, connected in a way that provides the device integrity, wherein said filtering component accommodates a filtering element and at least one sealing element.

50. Filtering device according to claim 49, wherein the cover, the filtering component and the recipient are connected by threads, pressure fits or grooves, or prominences.

51. Filtering device according to claim 49, wherein the cover has an upper end provided with a beak or orifice varying according to the instrument used to aspirate and/or introduce the marrow aspirate into the device, and a lower end which remains in contact with the filtering component.

52. Filtering device according to claim 49, wherein the filtering component comprises an upper cavity and a lower cavity which are formed by a tub, the base of which constitutes a wall provided with at least one opening.

53. Filtering device according to claim 49, wherein the filtering component comprises an upper cavity and a lower cavity which are formed by a wall provided with at least one opening.

54. Filtering device according to claim 49, wherein, by said higher cavity of the filtering element accommodating over the wall the filtering element, which is fixed by at least one sealing element.

55. Filtering device according to claim 52, wherein the dimension(s) of the upper and/or lower cavity(ies) and the filtering component may vary.

56. Filtering device according to claim 52, wherein the wall of the filtering element is provided with at least one opening, said at least one opening having a configuration selected from a circle, a rectangle, a triangle, and combinations thereof.

57. Filtering device according to claim 49, wherein the sealing element is located over and/or under the filtering element.

58. Filtering device according to claim 49, wherein the filtering element is fixed by two sealing elements, wherein the first element is assembled at the upper portion and the second element is assembled at the lower portion of the filtering element.

59. Filtering device according to claim 49, wherein the sealing element surrounds the filtering element.

60. Filtering device according to claim 49, wherein the filtering element is located between three sealing elements.

61. Filtering device according to claim 49, wherein the sealing element is made of a biocompatible, elastomeric material.

62. Filtering device according to claim 49, wherein the format of said sealing element is selected from powder, chips, granules, paste, tablets, or block forms or a mixture thereof.

63. Filtering device according to claim 49, wherein the filtering element presents a cylindrical, disk, parallelepiped, cubicle, prismatic, oval, or spherical form.

64. Filtering device according to claim 49, wherein the recipient has a cylindrical, rectangular, square, oval, spherical, or pyramidal shape.

65. Filtering device according to claim 49, wherein the recipient has an anatomic shape, with a strap or handle.

66. Filtering device according to claim 49, wherein said device comprises an upper chamber formed by the cavity of the cover and the upper cavity, and a lower chamber corresponding to the cavity of the recipient itself.

67. Filtering device according to claim 66, wherein the volume ratio between the chambers of the device is about 0.5:15.

68. Filtering device according to claim 49, wherein the recipient comprises at least an opening and a holding base, said holding base being cylindrical, rectangular, square or pyramidal.

69. Filtering device according to claim 68, wherein the ratio between the height of the recipient and its base is about 1:0.2 to 1:0.8.

70. Filtering device according to claim 49, wherein the device is disposable or reusable.

71. Filtering device according to claim 49, wherein the device is made of polycarbonate.

72. A kit for obtaining an enriched filtering element, comprising:
i) a filtering device for bone marrow aspirate according to claim 49; and
ii) a filtering element;
and, optionally:
iii) one or more bone marrow aspiration devices;
iv) at least a syringe;
v) a device for modeling the filtering element;
vi) a device for fractioning the filtering element; and
vii) an auxiliary device for placing and/or withdrawal of the filtering element.

73. A kit according to claim 72, wherein the bone marrow aspiration device is a set of a cannula and a trocar.

74. A method for obtaining a biocompatible graftable material enriched with progenitor cells from the bone marrow aspirate, comprising the steps of:
a) obtaining a bone marrow aspirate;
b) filtering the bone marrow aspirate by introducing it within a filtering device, containing a filtering element which comprises a biocompatible graftable material; and
c) withdrawing said filtering element; and
a reprocess of step (b).

75. An enriched biocompatible material, which is obtained according to the method of claim 74.
